(19) United States
(12) Patent Application Publication
(10) Pub. No.: US 2012/0040009 A1
(43) Pub. Date: Feb. 16, 2012

(54) PARTICULATE PHARMACEUTICAL COMPOSITION WITH AN OPIOID AND AN OPIOID ANTAGONIST

(76) Inventor: Lars Holger Hermann, Schindellegi (CH)

(21) Appl. No.: 13/265,860

(22) PCT Filed: Apr. 22, 2010
(86) PCT No.: PCT/DE2010/000453

§ 371 Date: Oct. 22, 2011

(30) Foreign Application Priority Data
Apr. 22, 2009 (DE) 10 2009 018 474.0

(51) Int. Cl. A61K 9/14; A61P 25/36; A61P 25/04; A61K 31/485

(52) U.S. Cl. 424/497; 514/282; 424/490

(57) ABSTRACT

The invention relates to a pharmaceutical composition comprising first particles and second particles, the first particles comprising at least one opioid or a pharmaceutically acceptable salt thereof, and the second particles comprising at least one opioid antagonist or a pharmaceutically acceptable salt thereof, wherein the first and second particles cannot be distinguished from one another by visually detectable and/or physical properties, wherein the release of the opioid antagonist occurs continuously over a period of 30 minutes to as much as 8 hours after oral administration, and a dosage form containing it for peroral administration. In addition, the invention relates to a pharmaceutical composition that comprises a particle with the opioid and with the opioid antagonist with the above-mentioned release characteristics.

The present invention relates to a pharmaceutical composition with an opioid, preferably morphine, and an opioid antagonist, preferably naloxone. In particular, the present invention relates to a pharmaceutical composition for the controlled release of the opioid antagonist in a particular region of the digestive system, specifically continuously over a period of 30 minutes to as much as 8 hours after oral administration. In addition, the present invention relates to a pharmaceutical composition that comprises first particles with the opioid and second particles with the opioid antagonist, wherein the first and second particles cannot be distinguished from one another. In addition, the present invention relates to a pharmaceutical composition that comprises a particle with the opioid and with the opioid antagonist.

### STATE OF THE ART

The group of opioids (“similar to opium”) is a chemically heterogeneous group of natural and synthetic substances which have properties similar to morphine and are effective at opioid receptors. A distinction is made between the body's own (endogenous) opioids, which play a role in suppressing pain in the context of the stress reaction, and (exogenous) opioids, which are applied for therapy or abuse. “Opiates” is the term used for the opioids that occur naturally in opium, which are alkaloids in chemical terms and which include morphine.

The powerful pain-relieving effect (analgesia) of opioids is of great therapeutic importance. In contrast to the non-opioid analgesics, opioids develop their analgesic effect primarily in the central nervous system (CNS). The commonest unwanted side-effects of opioids are nausea, vomiting, dizziness and, especially in the case of long-term use, (spastic) constipation of the intestine. Overdoses of opioids can lead to dangerous respiratory depression, which can even go as far as asphyxiation.

If opioids are used regularly over a lengthy period, the development of tolerance (habituation) can occur. As a result, higher and higher doses are needed in order to achieve the desired effects. This is a pharmacodynamic tolerance, which is mainly due to increased enzymatic activity of the intracellular adenylate cyclase.

Like benzodiazepines, opioids are among the substances that can cause powerful drug dependency. A distinction has to be made here between mental and physical dependency. The mental component of dependency is attributed above all to the anxiolytic and euphorisiant effects of opioids. Physical dependency is mainly due to the fact that when the application of the opioid is interrupted, withdrawal symptoms can occur, which result from an increased release of noradrenaline. Typical withdrawal symptoms are restlessness, sensation of pain without cause, depression, vomiting and stomach cramps, diarrhoea, exhaustion and influenza-like conditions.

Withdrawal from opioids is extremely protracted. While physical withdrawal is usually overcome quickly, sleep problems or nightmares can still occur a year later when large doses have previously been consumed regularly. Mental cravings mean that relapses frequently occur.

Among the opioids commonly used for therapeutic purposes are tilidine, tramadol and morphine. In the case of very severe pain, tilidine, for example, is frequently not sufficient, and morphine or morphine-like substances are used. Morphine (5R,6S,9R,13S,14R)-4,5-epoxy-N-methyl morphine-7-ene-3,6-diol), also referred to as morphium in common parlance, is a very effective analgesic which is obtained from the seedpods of the common poppy and has the following structural formula:

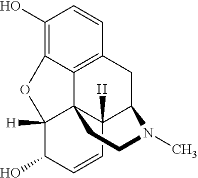

Among the opioids with the most powerful analgesic effect are sufentanil, remifentanil and fentanyl. Heroin (diacetyl morphine), the administration of which is prohibited in Germany, is of no therapeutic significance. In a heroin substitution therapy, the opioid methadone is mainly used.

Especially because of the psychotropic effect of opioids, there is a risk of improper application (abuse). One form of abuse is when the drug is not delivered by the administration route intended. Intravenous administration (injecting), for example, and nasal (sniffing) or pulmonary (smoking) routes are preferred to oral ingestion because of the rapid onset of action (“kick”). To this end, a preparation is melted or dissolved and injected intravenously, or it is burnt on aluminium film and the smoke inhaled (smoking on tin foil).

In order to limit the abusive application of opiods (protection against abuse), it is possible to mix opioid antagonists with the pharmaceutical preparations. One conventional, commercially available preparation of this kind is Valoron® N, which contains a combination of tilidine and naloxone. Naloxone (5R,9R,13S,14S)-17-allyl-3,14-dihydroxy-4,5-epoxymorphinan-6-on) is an opioid antagonist with the following structural formula:

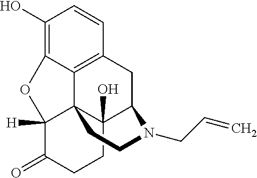

Together with naltrexone, naloxone is one of the pure opioid antagonists, which act as competitive antagonists at all the opioid receptors. Buprenorphine, on the other hand, acts as a mixed agonist/antagonist at the μ-receptor. The antagonistic effect cancels out the effect of the opioid, i.e. the agonist. This property is exploited when naloxone, for example, is used therapeutically as an antidote in the event of an opioid overdose.

In the case of oral administration, naloxone is subject to a high first-pass effect, i.e. substantial inactivation in the liver before it reaches the site of action. In the case of intravenous administration, on the other hand, a major portion of the naloxone remains effective. Since naloxone, as an opioid antagonist, cancels out the effect of the opiod, the simultaneous intravenous administration of an opioid and an opioid antagonist triggers a withdrawal syndrome. In the case of pulmonary, nasal, transdermal or rectal administration too, naloxone can develop its antagonistic effect.

One precondition for naloxone's performing its function as a protection against abuse is that the opioid antagonist cannot be readily separated from the opioid. That would be the case, for example, if the opioid and the opiod antagonist were administered with different tablets. In the case of multi-layer preparations, for instance, it is possible to dissolve the individual layers in solvent, such as water or ethanol, and to recover the opioid by evaporating the solution obtained. A dosage form with morphine containing a naltrexone core is known. It is possible to separate morphine and naltrexone by placing the dosage form in water for 20 minutes. After that, the morphine is dissolved and can be recovered by straining the naltrexone cores. Manual separation, for example by scratching off or breaking open the outer cover of a preparation is also possible.

WO 2007/082935 discloses dosage forms in which the opioid and the opioid antagonist are present as a mixture of powder or granules. In the mixture, the particles containing the opioid and those containing the opioid antagonist cannot be distinguished from one another visually. In addition, it is disclosed that the opioid and/or the opioid antagonist can be released in a delayed manner.

Although naloxone basically does not develop its desired negative effects in the event of oral administration, even oral ingestion is not without side-effects. If active naloxone reaches the colon of patients who have developed constipation as a consequence of the long-term use of opioids, it may cause severe diarrhoea, which may last for up to four weeks. Because of the risk that withdrawal symptoms may occur directly, taking Valoron® N when there is already an addiction to opiates, for example, is contra-indicated.

In addition, even in the case of oral administration, it is possible for naloxone to be absorbed, cirumventing the first pass effect. Studies have shown that naloxone is easily absorbed in the mouth. When absorbed through the mucous membranes in the oral cavity, such as through the mucous membrane of the tongue (perlingually) and the inner surfaces of the cheeks, an active agent passes via the venous blood from the oral mucosa directly into the superior vena cava. This principle is exploited in the case of sublingual forms of administration, for example. With rectal absorption too, the first-pass effect is at least partially circumvented, which is exploited in the case of the administration of drugs by means of suppositories.

Because of the side-effects in opioid-dependent patients, admixing naloxone to morphine when it is used to treat pain or as a substitution therapy is highly problematic. In an extreme case, the therapy has to be discontinued.

One problem of the invention is therefore to provide a more readily tolerated pharmaceutical composition with an opioid and an opioid antagonist. In particular, it is an object of the invention to provide an opioid composition which also allows application in the case of opioid dependence or opioid tolerance.

### BRIEF SUMMARY OF THE INVENTION

The problem of the invention is solved by a pharmaceutical composition comprising first particles and second particles, the first particles comprising at least one opioid or a pharmaceutically acceptable salt thereof, and the second particles comprising at least one opioid antagonist or a pharmaceutically acceptable salt thereof, wherein the first and second particles cannot be distinguished from one another by visually detectable and/or physical properties, characterized in that the release of the opioid antagonist occurs continuously over a period of 30 minutes to as much as 8 hours after oral administration.

The problem of the invention is further solved by a pharmaceutical composition comprising a particle that comprises at least one opioid or a pharmaceutically acceptable salt thereof, and at least one opioid antagonist or a pharmaceutically acceptable salt thereof, characterized in that the release of the opioid antagonist occurs continuously over a period of 30 minutes to as much as 8 hours after oral administration.

In one embodiment of the pharmaceutical compositions of the invention the release of the opioid antagonist occurs continuously over a period of 30 minutes to as much as 6 hours, preferably of 45 minutes to 4.5 hours, after oral administration.

In one embodiment, the opioid is a full agonist, preferably morphine.

In one embodiment, the opiod antagonist exhibits a bioavailability of less than 5% and is preferably naloxone.

In one embodiment, the release of the opioid occurs over a period of 0 to at least 12 hours, preferably of 0 to 24 hours, after oral administration.

In one embodiment, the first and second particles are pellets or the one particle is in the form of pellets.

In one embodiment, the pellets comprise a core and a layer coat for controlled release of the drug.

In one embodiment, the layer coat for the release of the active agent comprises at least a polyacrylate/polymethacrylate polymer, especially at least a Eudragit®.

The problem of the present invention is further solved by a dosage form for peroral administration, which comprises one of the pharmaceutical compositions of the present invention, for administration three times, preferably twice, particularly preferably once daily.

In one embodiment, the dosage form is a capsule, preferably a hard gelatine capsule, or a sachet.

In one embodiment, the ratio of opioid antagonist to opioid in the dosage form is a figure of less than 1:10, preferably a figure in the range from 1:250 to <1:10, and particularly preferably a figure from 1:100.

The problem of the invention is further solved by a pharmaceutical composition of the present invention for use in the treatment of opioid dependency.

In one embodiment of the composition, the dose of the opioid in the dosage form is 200 mg. The dose of the antagonist is accordingly preferably 2 mg.

The problem of the invention is further solved by a pharmaceutical composition of the present invention for use in the treatment of pain in patients with an opioid dependency or without an opioid dependency.

In one embodiment of the composition, the dose of the opioid in the dosage form is 30 or 60 mg. The dose of the antagonist is accordingly preferably 0.3 or 0.6 mg.

### DETAILED DESCRIPTION OF THE INVENTION

The opioid, preferably morphine, may be present as a physiologically acceptable salt such as hydrochloride, hydrate, sulphate or chlorate, or as quaternary salts. Preferred salts of morphine are morphine hydrochloride, morphine sulphate pentahydrate, morphine chlorate, morphine methobromide or other quaternary salts of morphine and morphine N-oxide. Morphine sulphate pentahydrate is particularly preferred.

The opioid antagonist, preferably naloxone, may be present as a physiologically acceptable salt such as hydrochloride or hydrochloride dihydrate. Naloxone hydrochloride dihydrate is particularly preferred.

The present invention provides more readily tolerated pharmaceutical compositions with an opioid and an opioid antagonist, with which the occurrence of withdrawal symptoms is avoided. This advantage is achieved by means of the particular release profile of the opiod and opioid antagonist and also thanks to the ratio of these two to one another. The effect of the release profile consists in the fact that, unlike the concept generally adopted so far, namely the avoidance of “total withdrawal”, “local partial withdrawal” in the gut is also avoided.

The pharmaceutical composition is formulated such that the opioid develops its advantageous effect, while the effect of the opioid antagonist is reduced considerably or cancelled out in the case of oral administration. This characteristic of the pharmaceutical composition, i.e. better tolerance, is achieved by the controlled release of the opioid antagonist, i.e. by the release of the opioid antagonist in a specific region of the digestive system. At the same time, the release of the opioid is delayed (delayed release, sustained-release formulation). Since the first-pass effect is circumvented in the case of absorption of the opioid antagonist in the oral cavity, it is advantageous to prevent absorption in this section of the digestive system. This is achieved in accordance with the invention be ensuring that, in the first 30 minutes after oral administration of the pharmaceutical composition (or in the presence of a physiological salt solution), virtually no release of the opioid antagonist occurs.

In addition, it is advantageous to prevent absorption of the opioid antagonist in the rectum. This is achieved in accordance with the invention be ensuring that 8 hours, preferably 6 hours, after oral administration of the pharmaceutical composition (or in the presence of a physiological salt solution), the opioid antagonist is released virtually completely, i.e. it has already been released when the pharmaceutical composition reaches the rectum.

As of the transverse colon, and specifically as of the descending colon, absorption of the opioid antagonist is no longer possible, or only to a slight extent, since the absence of liquid means that the latter can no longer dissolve and is still subject to the first-pass effect. Absorption does not occur again until the rectum. It is therefore an advantage that the opioid antagonist has already been released when the pharmaceutical composition reaches the end of the transverse colon, or the beginning of the descending colon. This is achieved in accordance with the invention be ensuring that 8 hours, preferably 6 hours, after oral administration of the pharmaceutical composition (or in the presence of a physiological salt solution), the opioid antagonist is released virtually completely, i.e. it has already been released when the pharmaceutical composition reaches that section of the digestive system after about 10 hours. If the antagonist is released in the rectum, it becomes fully effective, circumventing the first-pass effect, and causes general and local withdrawal.

Since an opioid antagonist can trigger violent diarrhoea in the colon in opioid-dependent patients, it is also advantageous to prevent release in this section of the digestive tract wherever possible. This is achieved in accordance with the invention be ensuring that 8 hours, preferably 6 hours, after oral administration of the pharmaceutical composition (or in the presence of a physiological salt solution), the opioid antagonist is released virtually completely, i.e. it has already been released when the pharmaceutical composition reaches the colon after about 6 hours.

A key feature of the invention is that the release of naloxone takes place continuously, i.e. that it is sustained release, in line with approximately first-order kinetics, and virtually no release peaks occur.

In the state of the art, various possibilities are known for achieving the specific release profile of the opioid or opioid antagonist. The currently preferred solution is one in which the release is controlled by means of a particular sustained-release coating, though the sustained-release coating proposed in the following examples is only intended as illustrative, since other possibilities are conceivable and can be derived from the state of the art by the person of average skill in the art. As an alternative to a sustained-release coating, an appropriate sustained-release matrix can also be chosen, from which the opioid or opioid antagonist is released.

Since in the embodiment with the first and second particles, in which the opioid or opioid antagonist is contained, the particles cannot be distinguished by visually detectable properties, such as colour, shape or size, or physically measurable properties, such as weight or density, easy separability of the two substances is avoided. If the particles should be very similar in size, this prevents separation by sieving. If they are very similar in weight or density, this prevents separation on the basis of different floating properties (skimming).

In the embodiment with only one type of particle, containing both the opioid and the opioid antagonist, easy separability of the two substances is likewise avoided.

Recent findings have shown that opioid-dependent patients manifest an elevated absorption of opioids and opioid antagonists compared to non-dependent patients (Halbsguth U., Rentsch K. M., Eich-Höchli D., Diterich I., Fattinger K. Br. J. Clin. Pharmacol. 66: 781-91, 2008). In these patients, a ratio of opioid to opioid antagonist of more than 1:10 triggers withdrawal symptoms. It is therefore advantageous if the ratio of opioid to opioid antagonist is less than 1:10.

In order to obtain protection against abuse, a high ratio of opioid to opioid antagonist has hitherto been regarded as advantageous or necessary. Valoron® N solution, for example, contains 4 mg naloxone and 50 mg tilidine in 0.72 ml, i.e. in a ratio of 1:12.5. Suboxone® contains 2 mg naloxone and 8 mg buprenorphine, i.e. a ratio of 1:4. It has, however, transpired that in the case of non-opioid-dependent patients, 0.4 mg naloxone is sufficient to trigger discrete withdrawal symptoms in the event of the intravenous administration of 200 mg morphine, i.e. at a ratio of naloxone to morphine of 1:500. A ratio of 1:100 is considered ideal, i.e. withdrawal symptoms are triggered with i.v. application, without life-threatening conditions arising. It has furthermore surprisingly been found that the half-lives of naloxone and morphine are more similar than hitherto supposed.

In addition to the above-described advantageous properties and effects of the pharmaceutical composition or dosage form in accordance with the invention, a study recently conducted has found another, hitherto unknown, positive effect of the ultra-low dosing of the antagonist, in this case naloxone, which is possible with this invention.

It is known from the literature that ultra-low doses of opiate antagonists have positive effects on the development of opiate tolerance and dependency, enhance an anti-nociceptive effect, have an opiate-saving effect and exhibit an effect on alcohol dependence.

In the above-mentioned study, it has now been shown that additional positive effects surprisingly occurred, namely a reduction in the side-effects typical of morphine, such as nausea and itching, a reduction in or avoidance of constipation, a reduction in opiate craving, an improvement in cognition (among the cognitive skills of a human being are, for example, attentiveness, memory, learning, creativity, planning, orientation, imagination, argumentation, introspection, will, faith, etc.) and emotional responsiveness (also referred to as affective responsiveness. Emotional responsiveness expresses an appropriate ability to modulate moods. This means that the emotions perceivable by the person concerned or by an observer adjust appropriately to the subject of the conversation and the contact situation and that the standard gamut of feelings can be expressed perceptibly. In assessments by outsiders, emotional responsiveness is also judged according to the facial reactions and gestures and the adaptation of the voice during conversational contact), the improvement in appetite and feelings of hunger, the reduction in the feeling of bloatedness and the normalisation of the day-and-night rhythm or sleep pattern. In patients who were also dependent on alcohol, a reduction in the effects of the withdrawal of alcohol, or of the craving for alcohol, was apparent with the composition or dosage form of the invention.

### DESCRIPTION OF THE DRAWING

FIG. 1 is a graphical representation of the release data of naloxone over time.

### EXAMPLE

Preparation of the Pharmaceutical Composition (with 2 Particles)

The pharmaceutical composition comprises a mixture of morphine and naloxone pellets. Each pellet contains a core on which the drug, i.e. either morphine or naloxone, is applied and a layer coat to control the release of the drug.

Morphine Pellets

First of all, spherical pellet cores (sugar spheres) are film-coated using a suspension containing morphine sulphate, povidone (Kollidon K 25) and titanium dioxide in purified water. The pellets loaded with morphine are then sprayed with a dispersion of colloidal, anhydrous silica (Aerosil 200) in purified water.

After that, a first layer, which delays the release, is applied. For this purpose, the pellets are film-coated using a Eudragit coating suspension I containing talcum and Eudragit FS 30 D in purified water.

After that, a second layer, which delays the release, is applied. For this purpose, hypromellose is dispersed in purified water, and polysorbate 80 is added. A dispersion of talcum and titanium dioxide is added to this solution. Then Eudragit NE 30 D and Eudragit FS 30 D are added. The pellets are film-coated using this Eudragit coating suspension II. A dispersion of colloidal, anhydrous silica is sprayed onto this layer.

Naloxone Pellets

First of all, spherical pellet cores (sugar spheres) are film-coated using a suspension containing naloxone hydrochloride dihydrate, povidone (Kollidon K 25) and titanium dioxide in purified water. These pellets loaded with naloxone are then sprayed with a dispersion of colloidal, anhydrous silica (Aerosil 200).

After that, a layer which delays the release is applied. For this purpose, hypromellose is dispersed in purified water, and polysorbate 80 is added. A dispersion of talcum and titanium dioxide is added to this solution. Then Eudragit NE 30 D and Eudragit FS 30 D are added.

The pellets are film-coated with this Eudragit coating suspension. A dispersion of colloidal, anhydrous silica is sprayed onto this layer, onto the pellets. The ratio of the components of the layer coat is adjusted such that the desired release profile is obtained.

Comparison of Morphine and Naloxone Pellets

Table 1 shows that the morphine and naloxone pellets have very similar properties with regard to, for example, their size (radius), density and weight.

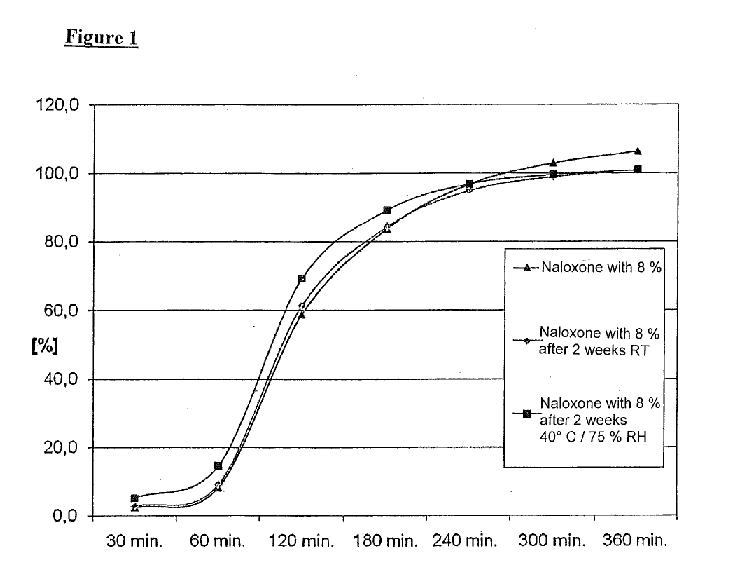

Table 2 and FIG. 1 show the course of the release of naloxone over a period of 360 minutes after various storage conditions.

The release of naloxone begins after between 30 and 60 minutes and is virtually completed after about 270 to 300 minutes (4.5 to 5 hours). It takes place continuously over the entire period without the occurrence of peaks (FIG. 1).

Table 3 shows the course of the release of naloxone and morphine over time. The pellets contained naloxone (hydrochloride dihydrate) in an amount of 1.2 to 2.1 mg/2 g or morphine (sulphate pentahydrate) in an amount of 120 to 210 mg morphine/2 g.

Whereas the release of naloxone after 6 hours is not less than 90%, about 30-70% of the morphine have been released after 8 hours, and a morphine release of no less than 90% is achieved after 20 hours.

The features of the invention disclosed in the above description, in the claims and in the drawing can be essential to implementing the invention in its various embodiments both individually and in any combination.

TABLE 1

| | | Morphine | Naloxone | |
|---|---|---|---|---|
| Pellets | | 1,700-2,000 | 1,700-2,000 | [μm] |
| Pellet radius | | 0.925 | 0.925 | [mm] |
| Vibration/bulk density | m | 175.9 | 175.9 | [g] |
| Bulk volume | V1 | 202.0 | 202.0 | [ml] |
| Back volume | V2 | 197.0 | 197.0 | [ml] |
| Bulk density | | 0.8708 | 0.8708 | [g/ml] |
| Tapped density | | 0.8929 | 0.8929 | [g/ml] |

$cp = \frac{π}{3 \cdot \sqrt{2}}$

| | | | | |
|---|---|---|---|---|
| Assumption of tightest sphere | cp | 0.7405 | 0.7405 | |
| packing | | | | |
| True density pellets | ρ | 1.2058 | 1.2058 | [g/ml] |

$V = \frac{4}{3} \cdot π \cdot {r}^{3}$

| | | | | |
|---|---|---|---|---|
| Volume | V | 3.315231098 | 3.315231098 | [mm³/Pellet] |

O = 4 · π · r²

| | | | | |
|---|---|---|---|---|
| Surface area | O | 10.75210086 | 10.75210086 | [mm²/pellet] |

TABLE 2

| | | 30 | 60 | 60. min. | 120 | 120 min. | 180 | 180 min. | 240 | 240 min. | 300 | 300 min. | 360 | 360 min. |
|---|---|---|---|---|---|---|---|---|---|---|---|---|---|---|
| | | min. | min. | corrected | min. | corrected | min. | corrected | min. | corrected | min. | corrected | min. | corrected |
| Naloxone | Sample 1 | 2.3 | 8.4 | 8.4 | 58.5 | 58.6 | 25.2 | 83.8 | 38.4 | 97.3 | 44.4 | 103.6 | 47.4 | 107.1 |
| with 8% | Sample 2 | 2.2 | 8.0 | 8.0 | 58.8 | 58.9 | 24.9 | 83.8 | 37.1 | 96.3 | 42.9 | 102.4 | 45.6 | 105.6 |
| | Average | 2.3 | 8.2 | 8.2 | 58.7 | 58.8 | 25.1 | 83.8 | 37.8 | 96.8 | 43.7 | 103.0 | 46.5 | 106.4 |
| Naloxone | Sample 1 | 2.6 | 8.7 | 8.7 | 60.5 | 60.6 | 21.8 | 82.4 | 32.2 | 93.0 | 36.0 | 97.2 | 37.4 | 98.9 |
| with 8% | Sample 2 | 2.8 | 9.1 | 9.1 | 61.1 | 61.2 | 24.0 | 85.2 | 34.3 | 95.8 | 38.1 | 99.9 | 39.3 | 101.5 |
| after 2 | Sample 3 | 2.9 | 9.4 | 9.4 | 62.2 | 62.3 | 23.3 | 85.6 | 33.3 | 95.9 | 37.0 | 99.9 | 38.3 | 101.6 |
| weeks RT | Average | 2.8 | 9.1 | 9.1 | 61.3 | 61.4 | 23.0 | 84.4 | 33.3 | 94.9 | 37.0 | 99.0 | 38.3 | 100.7 |
| Naloxone | Sample 1 | 5.2 | 14.0 | 14.1 | 67.8 | 68.0 | 20.3 | 88.3 | 28.0 | 96.2 | 30.6 | 99.1 | 31.6 | 100.4 |
| with 8% | Sample 2 | 5.5 | 15.5 | 15.6 | 69.6 | 69.8 | 19.4 | 89.2 | 26.8 | 96.8 | 29.1 | 99.4 | 30.0 | 100.6 |
| after 2 | Sample 3 | 5.1 | 13.9 | 14.0 | 69.8 | 70.0 | 20.1 | 90.1 | 27.4 | 97.6 | 29.9 | 100.4 | 30.8 | 101.6 |
| weeks | Average | 5.3 | 14.5 | 14.6 | 69.1 | 69.3 | 19.9 | 89.2 | 27.4 | 96.9 | 29.9 | 99.6 | 30.8 | 100.9 |
| 40° C./75% | | | | | | | | | | | | | | |
| RH | | | | | | | | | | | | | | |

TABLE 3
Calculation for ratio 100 to 1

| | | | |
|---|---|---|---|
| Start | End | Minimum released per hour | Maximum released per hour |

Controlled release of the antagonist naloxone

| | | | | |
|---|---|---|---|---|
| 0 | 0.5 | 0 | 0 | |
| 0.5 | 8 | * = 0.3 mg total/8 h* | * = 12 mg Total/8 h* | |
| | | 0.0375 | 1.5 | *mg/h |
| 8 | infinite | 0 | 0 | |

Controlled release of agonist morphine or others with less than 5% absorption after oral administration

| | | | | | |
|---|---|---|---|---|---|
| 0 | 2 | up to 20% | * = 30 mg total *20%/2 h* | * = 1,200 mg total *20%/2 h* | |
| 0 | 2 | | 3 | 120 | *mg/h |
| 2 | 8 | 20 to 70% | * = 30 mg total *50%/6 h* | * = 1,200 mg total *50%/6 h* | |
| 2 | 8 | | 2.5 | 100 | *mg/h |
| 8 | 12 | up to 50% | * = 30 mg total *50%/4 h* | * = 1,200 mg total *50%/4 h* | |
| 8 | 12 | | 3.75 | 150 | *mg/h |
| 0 | 24 | no less than 90% | * = 30 mg total *90%/24 h* | * = 1,200 mg total *90%/24 h* | |
| 0 | 24 | | 1.125 | 45 | *mg/h |

1. A pharmaceutical composition comprising first particles and second particles, the first particles comprising at least one opioid or a pharmaceutically acceptable salt thereof, and the second particles comprising at least one opioid antagonist or a pharmaceutically acceptable salt thereof, wherein the first and second particles cannot be distinguished from one another by visually detectable and/or physical properties, wherein the continuous release of the opioid antagonist begins at least 30 minutes after oral administration and ends not later than 8 hours after oral administration.

2. A pharmaceutical composition comprising a particle that comprises at least one opioid or a pharmaceutically acceptable salt thereof and at least one opioid antagonist or a pharmaceutically acceptable salt thereof, wherein the continuous release of the opioid antagonist begins at least 30 minutes after oral administration and ends not later than 8 hours after oral administration.

3. The pharmaceutical composition as claimed in claim 1, wherein the release of the opioid antagonist occurs from 30 minutes to as much as 6 hours, preferably from 45 minutes to 4.5 hours, after oral administration.

4. The pharmaceutical composition as claimed in claim 1, wherein the opioid is a full agonist.

5. The pharmaceutical composition as claimed in claim 4, wherein the opioid is morphine.

6. The pharmaceutical composition as claimed in claim 1, wherein the opioid antagonist is an opioid antagonist with an oral bioavailability of less than 5%.

7. The pharmaceutical composition as claimed in claim 6, wherein the opioid antagonist is naloxone.

8. The pharmaceutical composition as claimed in claim 1, wherein the release of the opioid occurs from 0 to at least 12 hours, preferably from 0 to 24 hours, after oral administration.

9. The pharmaceutical composition as claimed in claim 1, wherein the first and second particles are pellets or the one particle is in the form of pellets.

10. The pharmaceutical composition as claimed in claim 9, wherein the pellets comprise a core and a layer coat for controlled release of the drug.

11. The pharmaceutical composition as claimed in claim 10, wherein the layer coat for the controlled release of the active agent comprises at least a polyacrylate/polymethacrylate polymer.

12. The pharmaceutical composition as claimed in claim 11, wherein the layer coat for the controlled release of the active agent comprises at least a Eudragit®.

13. A dosage form for peroral administration, which comprises the pharmaceutical composition as claimed in claim 1, for administration twice, preferably once, daily.

14. The dosage form as claimed in claim 13, wherein the dosage form is a capsule, preferably a hard gelatine capsule, or a sachet.

15. The dosage form as claimed in claim 13, wherein the ratio of opioid antagonist to opioid is a figure of less than 1:10, preferably a figure in the range of 1:250 to <1:10, and particularly preferably a range of 1:100.

16. The dosage form as claimed in claim 13, wherein the dose of the opioid is 30, 60 or 200 mg.

17. A method of providing substitution therapy to an opiate addict, comprising:
diagnosing a patient as having opiate addiction,
preparing a pharmaceutical composition comprising first particles and second particles, the first particles comprising at least one opioid or a pharmaceutically acceptable salt thereof, and the second particles comprising at least one opioid antagonist or a pharmaceutically acceptable salt thereof, wherein the continuous release of the opioid antagonist begins at least 30 minutes after oral administration and ends not later than 8 hours after oral administration, wherein the opiate and opiate antagonist are present in a ratio which will alleviate withdrawal symptoms in an addict when taken orally but cause withdrawal symptoms in the addict when taken intravenously, and wherein said opiate and opiate antagonist are combined into an administration form that does not allow separation by mechanical or visual means or by differential solubility, and
administering the non-separable combination only orally to the addict for substitution therapy in an amount sufficient to treat the opiate dependency and prevent occurrence of withdrawal symptoms.

18. The method as claimed in claim 17, wherein the dose of the opioid is 200 mg.

19. (canceled)

20. The method as claimed in claim 17, wherein the dose of the opioid is 30 or 60 mg.

21. A method of providing pain therapy to an opiate dependent pain patient, comprising:
diagnosing a pain patient as having opiate dependency,
preparing a pharmaceutical composition comprising first particles and second particles, the first particles comprising at least one opioid or a pharmaceutically acceptable salt thereof, and the second particles comprising at least one opioid antagonist or a pharmaceutically acceptable salt thereof, wherein the continuous release of the opioid antagonist begins at least 30 minutes after oral administration and ends not later than 8 hours after oral administration, wherein the opiate and opiate antagonist are present in a ratio which will alleviate pain symptoms in an opiate dependent pain patient when taken orally but cause withdrawal symptoms in the opiate dependent pain patient when taken intravenously, and wherein said opiate and opiate antagonist are combined into an administration form that does not allow separation by mechanical or visual means or by differential solubility, and
administering the non-separable combination only orally to the opiate dependent pain patient for substitution therapy in an amount sufficient to prevent occurrence of withdrawal symptoms.

22. A method of providing pain therapy to an opiate dependent pain patient, comprising:
diagnosing a pain patient as having opiate dependency,
preparing a pharmaceutical composition comprising a particle that comprises at least one opioid or a pharmaceutically acceptable salt thereof and at least one opioid antagonist or a pharmaceutically acceptable salt thereof, wherein the continuous release of the opioid antagonist begins at least 30 minutes after oral administration and ends not later than 8 hours after oral administration, wherein the opiate and opiate antagonist are present in a ratio which will alleviate pain symptoms in an opiate dependent pain patient when taken orally but cause withdrawal symptoms in the opiate dependent pain patient when taken intravenously, and wherein said opiate and opiate antagonist are combined into an administration form that does not allow separation by mechanical or visual means or by differential solubility, and
administering the non-separable combination only orally to the opiate dependent pain patient for substitution therapy in an amount sufficient to prevent occurrence of withdrawal symptoms.

23. A method of providing substitution therapy to an opiate addict, comprising:
diagnosing a patient as having opiate addiction, preparing a pharmaceutical composition comprising a particle that comprises at least one opioid or a pharmaceutically acceptable salt thereof and at least one opioid antagonist or a pharmaceutically acceptable salt thereof, wherein the continuous release of the opioid antagonist begins at least 30 minutes after oral administration and ends not later than 8 hours after oral administration, wherein the opiate and opiate antagonist are present in a ratio which will alleviate withdrawal symptoms in an addict when taken orally but cause withdrawal symptoms in the addict when taken intravenously, and wherein said opiate and opiate antagonist are combined into an administration form that does not allow separation by mechanical or visual means or by differential solubility, and
administering the non-separable combination only orally to the addict for substitution therapy in an amount sufficient to treat the opiate dependency and prevent occurrence of withdrawal symptoms.
